(19) United States
(12) Patent Application Publication
(10) Pub. No.: US 2019/0201372 A1
(43) Pub. Date: Jul. 4, 2019

(54) LIPOSOMAL PREPARATION AND METHODS OF TREATMENT

(71) Applicant: HABI PHARMA PTY LTD, Crawley (AU)

(72) Inventor: William MCKAY, North Freemantle (AU)

(21) Appl. No.: 16/092,846

(22) PCT Filed: Apr. 7, 2017
(86) PCT No.: PCT/AU2017/050303

§ 371 Date: Oct. 11, 2018

(30) Foreign Application Priority Data
Apr. 12, 2016 (AU) 2016901363

(51) Int. Cl. A61K 31/352; A61K 9/127; A61P 25/08; A61K 31/05

(52) U.S. Cl. CPC A61K 31/352; A61K 31/05; A61P 25/08; A61K 9/1271

(57) ABSTRACT

Pharmaceutical preparations comprising cannabinoid resins encapsulated in liposomes are provided. The preparations are characterised by particular molar ratios of carboxyl containing cannabinoids to their decarboxylated counterparts. Also provided are methods of preparing the pharmaceutical preparations and their use in the treatment of disease or disorders.

### FIELD

The present disclosure relates generally to liposomal preparations for medicinal use and to methods of their preparation and particularly relates to liposomes encapsulating nano-particle emulsions comprising resin containing cannabinoids.

### BACKGROUND

The medicinal use of compounds including cannabinoids is known to provide therapeutic benefits in the treatment of many conditions. Cannabinoids and terpenes are compounds present in Cannabis plants, especially Cannabis Sativa and Cannabis Indica.

Some of the medical benefits attributable to one or more of the cannabinoids isolated from cannabis include treatment of pain, nausea, AIDS-related weight loss and wasting, multiple sclerosis, allergies, infection, depression, migraine, bipolar disorders, hypertension, post-stroke neuroprotection, epilepsy, and fibromyalgia, as well as inhibition of tumor growth, angiogenesis and metastasis. Studies have shown that cannabinoids may also be useful for treating conditions such as glaucoma, Parkinson's disease, Huntington's disease, migraines, inflammation, Crohn's disease, dystonia, rheumatoid arthritis, emesis due to chemotherapy, inflammatory bowel disease, atherosclerosis, posttraumatic stress disorder, cardiac reperfusion injury, prostate carcinoma, and Alzheimer's disease. For example, U.S. Pat. No. 6,630,507 discloses cannabinoids for use as antioxidants and neuroprotectants; U.S. Pat. No. 7,105,685 discloses cannabinoids for the treatment of diseases associated with immune dysfunction, particularly HIV disease and neoplastic disorders; U.S. Pat. No. 7,109,245 discloses cannabinoids useful as vasoconstrictors; United States Patent Application Publication No. US 2011/0257256 discloses tetrahydrocannabidiol cannabidiol (THC-CBD) composition for use in treating or preventing cognitive impairment and dementia; International Application Publication No. WO/2009/147439 discloses use of cannabinoids in the manufacture of a medicament for use in the treatment of cancer, International Patent Application Publication No. WO/2007/148094 discloses use of cannabinoid compositions for the treatment of neuropathic pain; and United States Patent Application Publication No. US 2010/0286098 discloses a method of treating tissue injury in a patient with colitis by administering cannabinoids.

It is also known to use a liposomal form of preparation to deliver drugs, as disclosed in United States Patent Application Publication no. US 2011/0064794 and International Patent Application Publication no. WO 2015/068052.

The treatment of some conditions benefit from the raw or un-heated phytocannabinoid components of the plant extract or resin, such as cannabidiolic acid (CBDA) and tetrahydrocannabinolic acid (THCA), whereas other conditions benefit from the heat treatment of the plant extract to convert some or most of these carboxylic acid forms of these components to their “decarboxylated” versions such as cannabidiol (CBD) and tetrahydrocannabinol (THC) by driving off carbon dioxide which removes a carbon atom. However many processes used to extract the plant extracts or resin and to formulate into a form that can deliver an easily measured and administered dose, heat the plant extract and change the profile of the compounds in a relatively uncontrolled way.

It would therefore be desirable to provide a method for formulating an easily measured and administered pharmaceutical preparation, the pharmaceutical preparation comprising a resin with a cannabinoid profile largely unaltered by the production of the pharmaceutical preparation.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### SUMMARY

According to one aspect of the present disclosure there is provided a pharmaceutical preparation comprising:
liposomes, said liposomes comprising an emulsified resin substantially encapsulated therein,
said emulsified resin comprising oil, water and at least one resin;
wherein the at least one resin comprises cannabinoids, and
wherein the molar ratio of at least one carboxyl containing cannabinoid to its decarboxylated form in the pharmaceutical preparation is greater than 1 to 10.

The molar ratio of at least one carboxyl containing cannabinoid to its decarboxylated form in the pharmaceutical preparation may be greater than 1 to 4, or greater than 1 to 2, or greater than 1 to 1, or greater than 2 to 1, or greater than 3 to 1, or greater than 4 to 1.

The molar ratio of cannabidiolic acid (CBDA) to cannabidiol (CBD) and/or the molar ratio of tetrahydrocannabinolic acid (THCA) to tetrahydrocannabinol (THC) in the pharmaceutical preparation may be greater than 1 to 10.

The molar ratio of CBDA to CBD may be greater than 1 to 4, or greater than 1 to 2, or greater than 1 to 1, or greater than 2 to 1, or greater than 3 to 1, or greater than 4 to 1.

The molar ratio of THCA to THC may be greater than 1 to 4, or greater than 1 to 2, or greater than 1 to 1, or greater than 2 to 1, or greater than 3 to 1, or greater than 4 to 1.

By substantially encapsulated it is meant that at least 80% by weight of the emulsified resin is encapsulated in the liposomes or at least 90% by weight of the emulsified resin is encapsulated in the liposomes.

At least 80% by weight of the liposomes may be less than 300 nm in size. Preferably at least 80% by weight of the liposomes may be less than 200 nm in size. More preferably at least 80% by weight of the liposomes may be less than 150 nm in size. The size of the liposomes may be determined by, for example, Dynamic Light Scattering or other techniques well known to those skilled in the art.

The liposomes may be PEGylated, through treatment with polyethylene glycol or functionalised derivatives thereof. Exemplary polyethylene glycols include polyethylene glycol (PEG) 400.

The emulsified resin may be a small particle emulsified resin in which at least 80% by weight of the particles are less than 150 nm. Preferably the particle size of the emulsified resin may be less than 100 nm. For example, at least 80% by weight of particles in the emulsified resin may be between 50 nm and 100 nm in size or at least 80% by weight of particles in the emulsified resin may be less than 100 nm. More preferably, at least 80% by weight of the particles in the emulsified resin may be less than 80 nm in size. The size of the particles in the emulsified resin may be determined by, for example, Dynamic Light Scattering or other techniques well known to those skilled in the art.

The emulsified resin may be PEGylated, through treatment with polyethylene glycol or functionalised derivatives thereof. Exemplary polyethylene glycols include polyethylene glycol (PEG) 400.

PEGylation may be performed prior to encapsulation in the liposomes, or through lipid layer(s), by PEGylation of the liposomes after encapsulation of particles of the emulsified resin.

The liposomes may be phospholipid-based liposomes. For example, the lipid may be or include lecithin.

The concentration of the emulsified resin in the preparation may be less than 3% by weight or by volume. Preferably the concentration of the emulsified resin in the preparation may be less than 2% by weight or volume and more preferably less than 1% by weight or volume.

The at least one resin may be a resin extracted from plant material. For example, the resin may be preferably extracted primarily from the flowers of a cannabis plant, particularly specific strains of cannabis plant. One significant advantage for using resin extracted from flowers, rather than synthetic cannabinoids, is to benefit from the documented entourage effect of the profile of (for example the amount of and balance between) naturally occurring compounds such as phytocannabinoids and terpenes found in combination in the plant material. This can, advantageously, produce a drug with improved medicinal benefits and/or less side-effects than synthetic cannabinoid drugs.

The at least one resin may include resins extracted from at least one plant. The at least one resin can, for example, be a blend of resins extracted from different strains of plants, or from the same strain of plant grown or harvested in different ways.

The oil may be an edible oil. The oil may be selected from the group consisting of corn, soybean, peanut, sunflower, rapeseed, olive, palm, rice bran and coconut oils.

The liposome particles may have the emulsified resin encapsulated within at least one lipid bilayer.

The pharmaceutical preparations may be characterised as having less than a 30%, more often less than a 20% alteration in the molar ratio of active components, such as cannabinoids and terpenes present in the resin extracted from the plant, prior to processing into a pharmaceutical preparation. This is advantageous as there is growing evidence that the naturally occurring carboxylic acid forms of some of the components of cannabis resin provide medicinal benefits not present in the decarboxylated forms.

The pharmaceutical preparation as herein disclosed may comprise any one or more of the hereinbefore disclosed features.

Another aspect of the present disclosure provides a method of producing the pharmaceutical preparation as hereinbefore described, the method comprising the steps of:
combining a resin, said resin comprising cannabinoids, with oil and water to form an emulsified resin;
combining the emulsified resin with a lipid; and
homogenising the lipid together with the emulsified resin to form liposomes;
   wherein the homogenisation is performed at a temperature at or below 60° C.

The temperature of homogenisation may be performed at or below 55° C., or at or below 50° C., or at or below 45° C., or at or below 40° C., or at or below 35° C., or at or below 30° C., or at or below 25° C., or at or below 20° C.

Another aspect of the present disclosure provides a method of producing the pharmaceutical preparation as hereinbefore described, the method comprising the steps of:
combining a resin, said resin comprising cannabinoids, with oil to form a resin-oil mixture;
combining the resin-oil mixture with water to form an emulsified resin;
combining the emulsified resin with a lipid; and
homogenising the lipid together with the emulsified resin to form liposomes;
   wherein the homogenisation is performed at a temperature at or below 60° C.

The temperature of homogenisation may be performed at or below 55° C., or at or below 50° C., or at or below 45° C., or at or below 40° C., or at or below 35° C., or at or below 30° C., or at or below 25° C., or at or below 20° C.

Another aspect of the present disclosure provides a method of producing the pharmaceutical preparation as hereinbefore described, the method comprising the steps of:
combining a resin, said resin comprising cannibinoids, with oil and water to form an emulsified resin;
combining the emulsified resin with a lipid; and
homogenising the lipid together with the emulsified resin using a pressure plate homogeniser to form liposomes.

The temperature of homogenisation may be performed at or below 55° C., or at or below 50° C., or at or below 45° C., or at or below 40° C., or at or below 35° C., or at or below 30° C., or at or below 25° C., or at or below 20° C.

Another aspect of the present disclosure provides a method of producing the pharmaceutical preparation as hereinbefore described, the method comprising the steps of:
combining a resin, said resin comprising cannibinoids, with oil to form a resin-oil mixture;
combining the resin-oil mixture with water to form an emulsified resin;
combining the emulsified resin with a lipid; and
homogenising the lipid together with the emulsified resin using a pressure plate homogeniser to form liposomes.

The temperature of homogenisation may be performed at or below 55° C., or at or below 50° C., or at or below 45° C., or at or below 40° C., or at or below 35° C., or at or below 30° C., or at or below 25° C., or at or below 20° C.

Another aspect of the present disclosure provides a method of producing a pharmaceutical preparation comprising liposomes, said liposomes comprising an emulsified resin substantially encapsulated in the liposomes, said emulsified resin containing cannabinoids, the method including the steps of:
combining a resin, said resin comprising cannibinoids, with oil and water to form an emulsified resin;
combining the emulsified resin with a lipid; and
homogenising the lipid together with the emulsified resin to form liposomes;
   wherein the homogenisation is performed at a temperature at or below 60° C.

The temperature of homogenisation may be performed at or below 55° C., or at or below 50° C., or at or below 45° C., or at or below 40° C., or at or below 35° C., or at or below 30° C., or at or below 25° C., or at or below 20° C.

Another aspect of the present disclosure provides a method of producing a pharmaceutical preparation comprising liposomes, said liposomes comprising an emulsified resin substantially encapsulated in the liposomes, said emulsified resin containing cannabinoids, the method including the steps of:
combining a resin, said resin comprising cannibinoids, with oil to form a resin-oil mixture;
combining the resin-oil mixture with water to form an emulsified resin;
combining the emulsified resin with a lipid; and
homogenising the lipid together with the emulsified resin to form liposomes;
   wherein the homogenisation is performed at a temperature at or below 60° C.

The temperature of homogenisation may be performed at or below 55° C., or at or below 50° C., or at or below 45° C., or at or below 40° C., or at or below 35° C., or at or below 30° C., or at or below 25° C., or at or below 20° C.

Another aspect of the present disclosure provides a method of producing a pharmaceutical preparation comprising liposomes, said liposomes comprising an emulsified resin substantially encapsulated in the liposomes, said emulsified resin comprising cannabinoids, the method including the steps of:
combining a resin, said resin comprising cannibinoids, with oil and water to form an emulsified resin;
combining the emulsified resin with a lipid; and
homogenising the lipid together with the emulsified resin using a pressure plate homogeniser to form liposomes.

The temperature of homogenisation may be performed at a temperature at or below 55° C., or at or below 50° C., or at or below 45° C., or at or below 40° C., or at or below 35° C., or at or below 30° C., or at or below 25° C., or at or below 20° C.

Another aspect of the present disclosure provides a method of producing a pharmaceutical preparation comprising liposomes, said liposomes comprising an emulsified resin substantially encapsulated in the liposomes, said emulsified resin comprising cannabinoids, the method including the steps of:
combining a resin, said resin comprising cannibinoids, with oil to form a resin-oil mixture;
combining the resin-oil mixture with water to form an emulsified resin;
combining the emulsified resin with a lipid; and
homogenising the lipid together with the emulsified resin using a pressure plate homogeniser to form liposomes.

The temperature of homogenisation may be performed at a temperature at or below 55° C., or at or below 50° C., or at or below 45° C., or at or below 40° C., or at or below 35° C., or at or below 30° C., or at or below 25° C., or at or below 20° C.

In any of the above disclosed methods the resin or similar material comprising cannabinoids may be obtained from the flowers of a cannabis plants.

In any of the above disclosed methods the oil may be edible oil.

In any of the above disclosed methods the water may be replaced with any other fluid capable of forming an emulsion with the oil.

In any of the above disclosed methods the liposome particles may have the emulsified resin encapsulated within at least one lipid bilayer.

In any of the above disclosed methods, the resin may have a profile of cannabinoids within a desired range. For example a profile of cannabinoids may comprise at least the percentages of some of the carboxylic acid forms of various cannabinoids in the resin and/or the percentages of some of the phenolic (decarboxylated) forms of various cannabinoids in the resin and/or the percentages of various terpenes in the resin.

In any of the above disclosed methods the resin may be obtained by extracting from cannabis flowers. The resin may have a profile of cannabinoids defined by one or more characteristics, such as the genetics of the plants, the growing conditions and/or the harvesting time, so the balance of the profile benefits from the entourage effect of multiple cannabinoids working in harmony.

In any of the above disclosed methods the resin may comprise mixing multiple resins (for example at least a first resin having a first profile of cannibinoids and a second resin having a second profile of cannibinoids). Additionally or alternatively, the resin-oil mixture may comprise at least a first resin-oil mixture and a second resin-oil mixture. For example, the step of combining water with the resin-oil mixture to form an emulsified resin may further comprise the step of combining the water and at least a first resin-oil mixture and a second resin-oil mixture.

In the above disclosed methods, the step of combining a resin, oil and water to form an emulsified resin may comprise the use of a sonic mixing device. Alternatively or additionally, the step of combining water with the resin-oil mixture to form an emulsified resin may comprise the use of a pressure plate homogeniser to mix the water and resin-oil mixture to an emulsion.

In the above disclosed methods, the step of combining water with the resin-oil mixture to form an emulsified resin may comprise the use of a sonic mixing device. Alternatively or additionally, the step of combining water with the resin-oil mixture to form an emulsified resin may comprise the use of a pressure plate homogeniser to mix the water and resin-oil mixture to an emulsion.

In the above disclosed methods the step of combining the resin, oil and water to form an emulsion (for example by using a pressure plate homogeniser) may be performed until at least 80% by weight of the particles in the emulsion are less than 100 nm in size. For example the emulsion may be passed past the pressure plate of a cold pressure plate homogeniser multiple times until the diameter of most of the particles is less than for example 100 nm or 80 nm. This may be achieved by processing for a specified or predetermined period of time or by measuring the particle size.

In the above disclosed methods the step of homogenising the resin-oil mixture together with the water to form an emulsion (for example by using a pressure plate homogeniser) may be performed until at least 80% by weight of the particles in the emulsion are less than 100 nm in size. For example the emulsion may be passed past the pressure plate of a cold pressure plate homogeniser multiple times until the diameter of most of the particles is less than for example 100 nm or 80 nm. This may be achieved by processing for a specified or predetermined period of time or by measuring the particle size.

In any of the above methods the emulsion may comprise at least a first emulsion containing a first resin and a second emulsion containing a second resin. For example, the step of combining a lipid together with the emulsion may comprise combining the lipid and at least a first emulsion and a second emulsion.

In any of the above methods the step of combining a lipid with the emulsion may comprise the step of utilising a sonic mixing device to mix the lipid and the emulsion together. Alternatively or additionally, the step of combining a lipid with the emulsion may comprise the step of utilising a vortex mixing device to mix the lipid and the emulsion together.

In any of the above methods the step of homogenising the lipid together with the emulsion using a pressure plate homogeniser may be performed until at least 80% by weight of the liposomes are less than 300 nm or less than 200 nm in size. For example the emulsion and lipid mixture may be passed past the pressure plate multiple times until the diameter of most of the particles is less than 200 nm. This may be achieved by processing for a specified or predetermined period of time or by measuring the particle size. Alternatively or additionally, the step of homogenising the lipid together with the emulsion using a pressure plate homogeniser may be performed using a cold pressure plate homogeniser. For example the homogeniser may have a cold water cooled pressure plate. Heating the pharmaceutical preparation can alter the profile of the active compounds (for example converting the cannabinoids CBDA to CBD and THCA to THC). Therefore it may be beneficial that the step of homogenising the lipid together with the emulsion using the pressure plate homogeniser be performed without increasing the temperature of the mixture above 60° C.

Any of the above disclosed methods of producing a pharmaceutical preparation may further comprise the step of PEGylating the emulsion and/or PEGylating the liposomes. For example, the step of PEGylating the liposomes may include adding polyethylene glycol (such as PEG400 which is approved for human consumption) to a liposomal mixture including the liposomes.

Any of the above disclosed methods may further comprise the step of encapsulating an amount of the liposomes in a capsule. For example, preferably a soft-shelled capsule can be filled with a known or measured amount of liposomal mixture containing the liposomes to give a known dose of the resin.

In another aspect there is provided a method for modulating cannibinoid receptors, particularly CB1 or CB2 receptors, in a subject, by the administration of an effective amount of a pharmaceutical preparation as disclosed herein.

In another aspect there is provided a method of treating a disease or disorder by the administration of an effective amount of a pharmaceutical preparation as disclosed herein, to a subject in need thereof.

In another aspect there is provided a method of treating a disease or disorder, said disease or disorder being associated with cannabinoid receptors modulation, particularly CB1 or CB2 receptors modulation, by the administration of an effective amount of a pharmaceutical preparation as disclosed herein, to a subject in need thereof.

In any of the above aspects, the disease or disorder may be selected from the group consisting of ocular pain and inflammation (glaucoma), AIDS wasting and other weight loss such as due to chemotherapy, neuropathic pain, somatic pain, spasticity associated with multiple sclerosis, fibromyalgia, movement disorders including dystonia, Parkinson's disease, Huntingdon's disease, Tourette's syndrome, muscular dystrophy, chemotherapy induced nausea, allergies, inflammation, infection, epilepsy, depression, chronic pain, migraine, Alzheimer's disease, bipolar disorders, anxiety disorder, inflammatory bowel disease, sleep disorders, restless legs syndrome, thyroid disorders, post-traumatic stress disorder, drug dependency and withdrawal and nausea.

In another aspect there is provided a method of treating pain by the administration of an effective amount of a pharmaceutical preparation as disclosed herein, to a subject in need thereof.

In another aspect there is provided a method of treating an immune disorder or an inflammatory disorder by the administration of an effective amount of a pharmaceutical preparation as disclosed herein, to a subject in need thereof.

In another aspect there is provided a method of treating epilepsy by the administration of an effective amount of a pharmaceutical preparation as disclosed herein, to a subject in need thereof.

In another aspect there is provided the use of a pharmaceutical preparation as disclosed herein in the manufacture of a medicament for the treatment of ocular pain and inflammation (glaucoma), AIDS wasting and other weight loss such as due to chemotherapy, neuropathic pain, somatic pain, spasticity associated with multiple sclerosis, fibromyalgia, movement disorders including dystonia, Parkinson's disease, Huntingdon's disease, Tourette's syndrome, muscular dystrophy, chemotherapy induced nausea, allergies, inflammation, infection, epilepsy, depression, chronic pain, migraine, Alzheimer's disease, bipolar disorders, anxiety disorder, inflammatory bowel disease, sleep disorders, restless legs syndrome, thyroid disorders, post-traumatic stress disorder, drug dependency and withdrawal and nausea.

In another aspect there is provided the use of a pharmaceutical preparation as disclosed herein in the manufacture of a medicament for the treatment of pain.

In another aspect there is provided the use of a pharmaceutical preparation as disclosed herein in the manufacture of a medicament for the treatment of an immune disorder or an inflammatory disorder.

In another aspect there is provided the use of a pharmaceutical preparation as disclosed herein in the manufacture of a medicament for the treatment of epilepsy.

In any of the above aspects the subject may be a mammal. In any of the above aspects the subject may be a human.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### BRIEF DESCRIPTION OF THE DRAWING

FIG. 1 is a flow diagram of a method of producing the pharmaceutical preparation according to an embodiment of the present disclosure.

### DESCRIPTION OF EXEMPLARY EMBODIMENTS

Before the present preparations and/or methods are disclosed and described, it is to be understood that unless otherwise indicated the present disclosure is not limited to specific preparations, components, methods, or the like, as such may vary, unless otherwise specified. It is also to be understood that the terminology used herein is for the purpose of describing particular embodiments only and is not intended to be limiting.

It is to be understood that while the present disclosure has been described in conjunction with the specific embodiments thereof, the foregoing description is intended to illustrate and not limit the scope of the disclosure. Other aspects, advantages and modifications will be apparent to those skilled in the art to which the disclosure pertains. Therefore, the following examples are put forth so as to provide those skilled in the art with a complete disclosure and description of how to make and use the disclosed preparations, and are not intended to limit the scope of the disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

All documents cited are herein fully incorporated by reference for all jurisdictions in which such incorporation is permitted and to the extent such disclosure is consistent with the description of the present disclosure.

The pharmaceutical preparations of the present disclosure may deliver a cannabinoid comprising resin extracted from the Cannabis plant. The resin is formed into an emulsion which is then substantially encapsulated inside liposomes. If required, the preparation can be made with less than a 30%, more often less than a 20% alteration in the profile of active components, such as cannabinoids and terpenes, present in the resin extracted from the plant prior to processing into a pharmaceutical preparation. This is possible due to the avoidance of excessive heat generating processes and is important in preventing the unintentional, uncontrolled and/or undesirable decarboxylation of the resin, driving off a carbon atom from the carboxylic acid forms of some of the components in the resin. Such decarboxylation is due to heating of the resin and converts components such as cannabidiolic acid (CBDA) and tetrahydrocannabinolic acid (THCA) into their phenolic versions cannabidiol (CBD) and tetrahydrocannabinol (THC). While it has been common practice to decarboxylate cannabis resin, there is growing evidence that the carboxylic acid forms of some of the components of cannabis resin provide medicinal benefits not present in the decarboxylated form.

Both the emulsion and the liposomes may be homogenised to reduce the respective particle sizes, increasing the efficiency of the delivery by both giving better penetration when ingested and better penetration into cells or across the blood-brain barrier or better interaction with cannabinoid receptors such as the CB1 receptors found primarily in the brain and the CB2 receptors found primarily in the immune system, although both CB1 and CB2 receptors may be found elsewhere in the body of humans and other mammals. The liposomal preparation of the present disclosure may be absorbed quickly, with absorption starting orally, for example under the tongue, and continuing through the digestive system.

The size of individual particles of the emulsion is preferably between 50 and 100 nanometres (nm) or between 50 and 80 nm in order to improve the desired efficiency of delivery. Although when preparing a homogenised emulsion it is not likely that every single particle will be in the desired range, at least 80% by weight of particles of the emulsion may, advantageously, be in the 50-80 nm range or 50-100 nm range. For example, some particles may coagulate and some may not be broken down within a 50-100 nm size range, but it is preferable that around 80% by weight of the emulsion particles containing resin are less than 100 nm in size to maintain efficient absorption of the cannabinoids of the preparation by the receptors.

The individual liposome size is ideally between 200 and 300 nm in order to hold and protect the emulsion and pass through the gut wall into the bloodstream. Once in the bloodstream, the liposomal nanoparticles break down through the dissolving of the lipid fat, leaving the emulsion nanoparticles floating in the bloodstream for delivery to the receptor sites. Using ultrasonic or vortex type homogenisers generate significant amounts of heat when used to break the liposome particles down to the desired less than 300 nm size. The levels of heat to which these ultrasonic and vortex processes subject the preparation cause the profile of compounds to be changed by providing a decarboxylating function.

Another improvement to the efficiency, or bio-availability of the pharmaceutical preparation may be obtained by using polyethylene glycol (PEG) to increase the half-life in the body. This is through reducing the rate at which the reticuloendothelial system clears the preparation. The emulsion can be PEGylated and/or the Liposomes can be PEGylated. Indeed PEGylating the liposomal mixture can result in some PEGylation of the emulsion particles inside the liposomes, PEGylation of the emulsion particles being more important than PEGylation of the liposomes. This PEGylation, together with the use of emulsion nanoparticles of less than 100 nm in size encapsulated in liposomes of less than 300 nm in size improves the bodies take up of the resin by approximately 5 times compared to ingesting the equivalent amount of resin in oil for example.

The pharmaceutical preparation also has the advantage that the resin is diluted to a level where accurate dosage administration is possible, particularly since the amount of resin required is low due to the above noted efficiencies of delivery. For example, there may be 1 gram or 1 ml of resin in 150 ml of the preparation and the dosage rate can for example be 15 micro-litres of such a preparation per kilogram of body weight, i.e. 0.15 ml per 10 kg. So ideally the preparation is less than 1% resin, but can be made more concentrated, for example 2% or even 3% resin. Alternatively a more concentrated preparation may contain 1 ml of resin in 57 ml of the preparation, or for more accurate dosage control a less concentrated preparation may be made having for example 1 ml of resin in 300 ml of preparation.

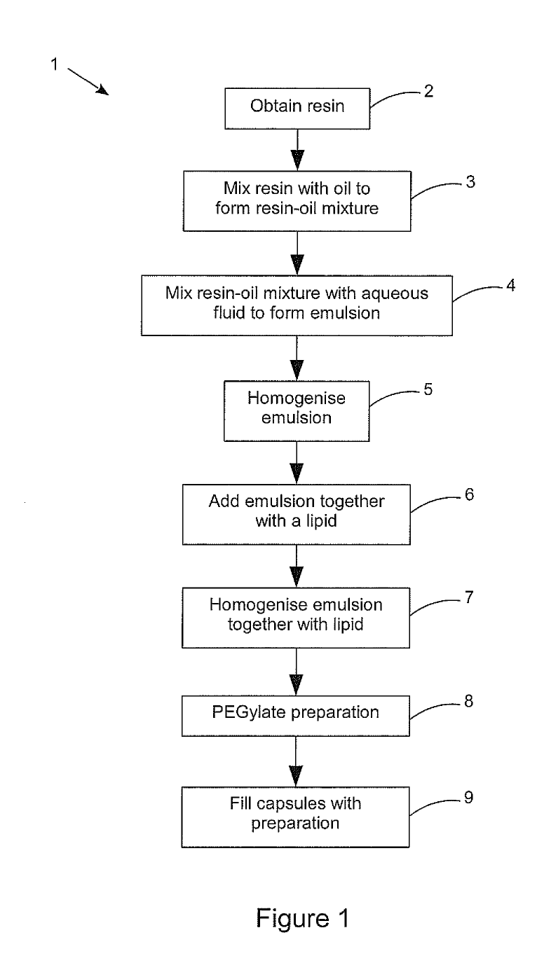

Referring now to FIG. 1, a process 1 is shown for producing the pharmaceutical preparation according to an embodiment of the present disclosure. The first step 2 is to obtain the resin to be encapsulated. If the carboxylic acid components of the cannabis extract used are required, the resin should be extracted cold, for example without use of significant amounts of heat to drive off solvent. CO₂ extraction processes are popular processes for cold extraction, such as using supercritical CO₂ under pressure, or using expanded CO₂ to supercool the resin which can then be vibrated off the other plant matter. Alternatively if the compounds are required to be decarboxylated to obtain the balance of CBDA to CDB or THCA to THC desired for example this can be done prior to processing into a liposomal form. For example it may be that the desired balance of CBDA to CBD in the final preparation is 1:10, 1:4, 1:2, 1:1, 2:1, 3:1 or 4:1, all of which are possible using the method of the present disclosure. Similarly if the balance of compounds required can be more closely achieved by blending two or more resins, this can be done in this initial obtain resin step. The resins can be from multiple strains of Cannabis plant, or from similar strains that have been grown, harvested and/or extracted differently. Ideally the required balance of compounds is obtained using extracts from primarily the flowers of Cannabis plants without the addition of synthetic cannabinoid compounds. The benefits of using the whole extract or resin from a plant are becoming better understood and are thought to be from the entourage effect of multiple compounds that occur naturally in combination. Even then, resins from genetically different strains having similar levels of the major compounds can have remarkably different levels of benefits.

As the resin has a very thick consistency, the next step 3 mixes an edible oil with the resin to make it possible to use in the following steps. For a given amount of resin, there may be an optimum amount of oil to add to form the desired resin-oil mixture, for example one part resin to one part oil by weight (i.e. 1:1). Again the resin used can be from one or more sources (different plant strains and or treatment) to achieve the desired profile of compounds.

At the next step 4, the resin-oil mixture is combined with an aqueous fluid (which can simply be water, but can include additives) to form an emulsion. Again the amount of resin-oil mixture to the amount of aqueous fluid is known to ensure the concentration of the final preparation is accurate. For example one part resin-oil mixture to 10 parts water by weight (i.e. 1:10). Again the resin-oil mixture used can be a blend of different resin-oils using resins with different characteristics (the profile of compounds). Low power sonic mixing may be used to form the emulsion as long as the power is kept low enough to avoid significant heating of the preparation at this stage. Ideally the emulsion is then homogenised down to particle sizes generally in the 50 to 100 nm range, i.e. at least 80% by weight of the particles in the emulsion are less than 150 nm in size. This level of homogenisation is ideally carried out using a pressure plate homogeniser as indicated in step 5 to minimise the heating of the preparation. Preferably the homogeniser is a cold pressure plate homogeniser, that is having a cooled pressure plate, such as one chilled by an internal flow of cold water. This first homogenising step, while optional, does enable the final preparation to achieve the desired particle size more readily. The cold pressure plate homogeniser is a continuous flow device, so the preparation may be passed multiple times past the pressure plate to reduce the particle size to the desired range. A suitable surfactant can optionally be used.

In the next step 6, the homogenised emulsion is added together with a lipid, typically a phospholipid such as lecithin, for example one part emulsion to 5.82 parts lipid by weight (i.e. 11:64). The example dilution rates given provide in total a concentration of 1 gram of resin in 150 ml of preparation allowing very accurate dosing. Again, to initially mix the emulsion and lipid together low power sonic mixing can be employed, or even in this case a vortex homogeniser can be used briefly to mix. However using ultrasonic homogenising to break the liposome particle size down to less than 200 nm would require the input of significant power and inevitably the heating of the preparation. Similarly using the vortex homogeniser to continue to homogenise the preparation and break down the liposome size to less than 200 nm would generate too much heat into the preparation in a poorly controlled manner, especially since most vortex homogenisers cannot get the particle size below 300 nm during a time that heats the preparation by an unacceptable amount. Therefore the homogenising of the emulsion and lipid may be included as an additional step 7 following the initial mixing of the two in step 6. Preferably the preparation of the emulsion and lipid is homogenised using a pressure plate homogeniser, preferably a cold pressure plate homogeniser. Although the homogenised preparation does not need to be tested for particle size continuously, the length of homogenisation time and/or number of passes of the preparation required can be found through testing and verified through periodic sample Liposome size checking.

The lecithin may be any known lecithin such as from soy or from sunflower oil. Lecithin from fractionated coconut oil may provide a preparation of above average stability without requiring preservatives, so preferably the lecithin used is a coconut oil extracted lecithin.

The preparation is preferably PEGylated as shown in step 8, typically using polyethylene glycol 400, to reduce the clearance rate of the preparation by the body's reticuloendothelial system as discussed above.

In the final optional step 9, the preparation may be used to fill capsules for administration. Although the use of a liposomal delivery format makes the administration of the liquid preparation relatively palatable, it can be preferable in some situations or for some users to use a capsule delivery system.

In the above description, the term terpene is intended to include any terpenoid and the term cannabinoid is intended to include any cannabinoid including synthetic cannabinoids, but preferably only the natural plant phytocannabinoids are used.

The pharmaceutical preparations as disclosed herein may be formulated into solid dosage forms for oral administration include capsules, dragees, tablets, pills, powders and granules. In such solid dosage forms, the pharmaceutical preparation may be mixed with at least one inert, pharmaceutically acceptable carrier or excipient, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions that can be used include polymeric substances and waxes.

The pharmaceutical compositions as disclosed herein can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the herein disclosed pharmaceutical preparation, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the herein disclosed pharmaceutical compositions, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Actual dosage levels of active ingredients in the pharmaceutical preparations as herein disclosed can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated.

When used in the herein disclosed or other treatments, a therapeutically effective amount of one of the compounds of the present disclosure contained within the pharmaceutical preparation can be employed in pure form or, where such forms exist, in pharmaceutically acceptable salt, ester or prodrug form. The phrase “therapeutically effective amount” of the compounds of the present disclosure means a sufficient amount of the compound to treat disorders, at a reasonable benefit/risk ratio applicable to any medical treatment. It will be understood, however, that the total daily usage of the compounds and preparations of the present disclosure will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

If desired, the effective daily dose can be divided into multiple doses for purposes of administration; consequently, single dose compositions may contain such amounts or submultiples thereof to make up the daily dose.

A dose may be given as 0.5 ml to 4 ml, or 0.5 ml to 3 ml, or 0.75 ml to 2.5 ml or 1 ml to 2 ml of pharmaceutical preparation. An adult dose may be less than 4 ml, or less than 3 ml, or less than 2 ml, or less than 1 ml of pharmaceutical preparation.

A dose of pharmaceutical preparation per kilogram (kg) of body weight may be in the range of 5 to 30 microlitres/kg, or 7.5-30 microlitres/kg. A dose of pharmaceutical preparation per kilogram (kg) of body weight may be in the range of less than 30 microlitres/kg, or less than 20 microlitres/kg, or less than 15 microlitres/kg, or less than 10 microlitres/kg.

1-37. (canceled)

38. A pharmaceutical preparation comprising:
liposomes, said liposomes comprising an emulsified resin substantially encapsulated therein,
said emulsified resin comprising oil, water and at least one resin;
wherein the at least one resin comprises cannabinoids, and
wherein the molar ratio of at least one carboxyl containing cannabinoid to its decarboxylated form in the pharmaceutical preparation is greater than 1 to 10; and
wherein the at least one resin is a resin extracted from plant material.

39. A pharmaceutical preparation as claimed in claim 38, wherein the liposomes are PEGylated.

40. A pharmaceutical preparation as claimed claim 38, wherein the emulsified resin is PEGylated.

41. A pharmaceutical preparation as claimed in claim 38, wherein the molar ratio of cannabidiolic acid (CBDA) to cannabidiol (CBD) and/or the molar ratio of tetrahydrocannabinolic acid (THCA) to tetrahydrocannabinol (THC) in the pharmaceutical preparation is greater than 1 to 10.

42. A pharmaceutical preparation as claimed in claim 38, wherein the molar ratio of cannabidiolic acid (CBDA) to cannabidiol (CBD) and/or the molar ratio of tetrahydrocannabinolic acid (THCA) to tetrahydrocannabinol (THC) in the pharmaceutical preparation is greater than 1 to 1.

43. A pharmaceutical preparation as claimed in claim 38, wherein at least 80% by weight of the emulsified resin is encapsulated in the liposomes.

44. A pharmaceutical preparation as claimed in claim 38, wherein at least 80% by weight of the liposomes have a particle size less than 300 nm.

45. A pharmaceutical preparation as claimed in claim 38, wherein the emulsified resin is a small particle emulsified resin in which at least 80% by weight of the particles have a particle size less than 150 nm.

46. A pharmaceutical preparation as claimed in claim 38, wherein the liposomes are phospholipid based liposomes.

47. A pharmaceutical preparation as claimed in claim 38, wherein the concentration of the at least one resin in the preparation is less than 3% by weight.

48. A pharmaceutical preparation as claimed in claim 38, wherein the at least one resin comprises resins extracted from more than one plant.

49. A method of producing the pharmaceutical preparation as claimed in claim 38, the method comprising the steps of:
combining a resin, said resin comprising cannibinoids, with oil and water to form an emulsified resin;
combining the emulsified resin with a lipid; and
homogenising the lipid together with the emulsified resin to form liposomes;
wherein the homogenisation is performed at a temperature at or below 60° C.

50. A method of producing the pharmaceutical preparation as claimed in claim 38, the method comprising the steps of:
combining a resin, said resin comprising cannibinoids, with oil and water to form an emulsified resin;
combining the emulsified resin with a lipid; and
homogenising the lipid together with the emulsified resin using a pressure plate homogeniser to form liposomes.

51. A method for modulating CB1 or CB2 receptors in a subject, by the administration of an effective amount of a pharmaceutical preparation as claimed in claim 38.

52. A method of treating a disease or disorder by the administration of an effective amount of a pharmaceutical preparation as claimed in claim 38, to a subject in need thereof.

53. A method as claimed in claim 52, wherein the disease or disorder is selected from the group consisting of ocular pain and inflammation (glaucoma), AIDS wasting and other weight loss such as due to chemotherapy, neuropathic pain, somatic pain, spasticity associated with multiple sclerosis, fibromyalgia, movement disorders including dystonia, Parkinson's disease, Huntingdon's disease, Tourette's syndrome, muscular dystrophy, chemotherapy induced nausea, allergies, inflammation, infection, epilepsy, depression, chronic pain, migraine, Alzheimer's disease, bipolar disorders, anxiety disorder, inflammatory bowel disease, sleep disorders, restless legs syndrome, thyroid disorders, post-traumatic stress disorder, drug dependency and withdrawal and nausea.

54. A method of treating pain by the administration of an effective amount of a pharmaceutical preparation as claimed in claim 38, to a subject in need thereof.

55. A method of treating an immune disorder or an inflammatory disorder by the administration of an effective amount of a pharmaceutical preparation as claimed in claim 38, to a subject in need thereof.

56. A method of treating epilepsy by the administration of an effective amount of a pharmaceutical preparation as claimed in claim 38, to a subject in need thereof.
